(19) United States
(12) Patent Application Publication
(10) Pub. No.: US 2004/0019235 A1
(43) Pub. Date: Jan. 29, 2004

(54) Method for producing higher (meth)acrylic acid esters

(76) Inventors: Friedrich-Georg Martin, Heidelberg (DE); Gerhard Nestler, Wien (AU); Jurgen Schroder, Ludwigshafen (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/433,612

(22) PCT Filed: Dec. 13, 2001
(86) PCT No.: PCT/EP01/14636

(30) Foreign Application Priority Data
Dec. 18, 2000 (DE) 100 63 175.4
Oct. 19, 2001 (DE) 101 52 680.6

(51) Int. Cl. C07C 69/52

(52) U.S. Cl. 560/205

(57) ABSTRACT

Higher (meth)acrylates are prepared by reacting (meth)acrylic acid and the alcohol in the presence of at least one acidic catalyst and at least one polymerization inhibitor and in the presence of a solvent which forms an azeotropic mixture with water and distilling off and condensing the azeotropic mixture, the condensate separating into an aqueous phase and an organic phase, by a process in which

a) the esterification is carried out in a reactor having a circulation evaporator and

b) in the presence of at least 10% by weight, based on the reaction mixture, of solvent, and either

c1) at least a part of the solvent, if required a part of the catalyst (mixture) and, if required, at least a part of the polymerization inhibitor (mixture) are initially taken in the esterification reactor and heated to the boiling point of the component having the lowest boiling point in the system and, as soon as the circulation is in operation, (meth)acrylic acid and alcohol and, if required, residual catalyst, polymerization inhibitor and solvent are metered in together or separately, or

c2) at least a part of the solvent, if required a part of the catalyst (mixture), if required at least a part of the polymerization inhibitor (mixture) and at least a part of the alcohol are initially taken in the esterification reactor and heated to the boiling point of the component having the lowest boiling point in the system and, as soon as the circulation is in operation, (meth)acrylic acid and, if required, the remaining alcohol and, if required, residual catalyst, polymerization inhibitor and solvent are metered in together or separately, or

c3) at least a part of the solvent, at least a part of the catalyst (mixture), at least a part of the polymerization inhibitor (mixture) and at least a part of the (meth)acrylic acid are initially taken in the esterification reactor and heated to the boiling point of the component having the lowest boiling point in the system and, as soon as the circulation is in operation, the alcohol and, if required, the remaining (meth)acrylic acid and, if required, residual catalyst, polymerization inhibitor and solvent are metered in together or separately, or

c4) at least a part of the solvent, at least a part of the catalyst (mixture), at least a part of the polymerization inhibitor (mixture), at least a part of the alcohol and a part of the (meth)acrylic acid are initially taken in the esterification reactor and heated to the boiling point of the component having the lowest boiling point in the system and, as soon as the circulation is in operation, if required the remaining alcohol and the remaining (meth)acrylic acid and, if required, residual catalyst, polymerization inhibitor and solvent are metered in together or separately.

The present invention describes a process for the preparation of higher (meth)acrylates by esterification of (meth)acrylic acid with the corresponding alcohols in the presence of at least one acidic catalyst, a polymerization inhibitor/polymerization inhibitor mixture and a solvent which forms an azeotropic mixture with water.

The novel process is suitable for the preparation of higher esters of (meth)acrylic acid with higher monohydric or polyhydric alcohols, polyether alcohols or polyester alcohols. Preferably, however, it can be used for the preparation of higher (meth)acrylates which have a molecular weight of >200 g/mol. Such esters cannot be purified by distillation.

Owing to their reactive double bond, higher (meth)acrylates are useful monomers which are used, for example, as coating raw materials for electron beam curing or as a component of UV-curable printing inks, surface coating materials, molding materials or casting materials or in adhesives.

In particular, colorless products without a natural odor and with a low acid number and long shelf life are required.

The preparation of higher (meth)acrylates by acid-catalyzed esterification of (meth)acrylic acid with the corresponding alcohols in the presence of an inhibitor/inhibitor system and, if required, of a solvent, e.g. benzene, toluene or cyclohexane, is generally known.

The catalysts used are as a rule sulfuric acid, arylsulfonic acids or alkanesulfonic acids or mixtures thereof.

Since the formation of the ester from (meth)acrylic acid and alcohol is known to be based on an equilibrium reaction, as a rule one feedstock is used in excess and/or the resulting water of esterification and/or the desired ester are removed from the equilibrium in order to obtain economical conversions. Influencing of the esterification equilibrium by the use of an excess of alcohol is, however, disadvantageous since this promotes, inter alia, the formation of ethers from the starting alcohols and of Michael adducts (cf. for example U.S. Pat. No. 4,280,010, column 1).

Michael adducts are understood as meaning products which are formed by addition of alcohols or (meth)acrylic acid at the double bond of (meth)acrylic compounds, e.g. alkoxypropionic acids or acryloyloxypropionic acids, and the esters thereof.

Since, owing to their high boiling points, the higher (meth)acrylates cannot as a rule be purified by distillation, these byproducts remain in the desired ester and influence the further processing and/or the quality of the subsequent products.

In the preparation of the higher (meth)acrylates, the water of reaction is as a rule therefore removed and in general an excess of (meth)acrylic acid is used. The water of esterification is usually separated off by distillation, by stripping, for example with air, or with the aid of a solvent which forms an azeotropic mixture with water.

Since (meth)acrylic compounds generally and polyfunctional (meth)acrylates in particular readily tend to undergo undesired polymerization, in particular under the action of heat, in general considerable efforts have been made to avoid the formation of polymer during the esterification and the isolation of the desired ester.

As a rule, this polymer formation does in fact lead to coating of the reactor walls, heat exchanger surfaces and column trays (fouling) and to blockage of pipes, pumps, valves, etc. (EP-A 522 709, page 2, lines 9-18; U.S. Pat. No. 5,171,888, column 1, lines 19-38). This results in expensive shutdowns and complicated cleaning operations, for example boiling with basic solutions, which subsequently have to be disposed of in an expensive manner, as described in DE-A 195 36 179.

The polymer in the reaction mixture moreover hinders working-up by causing phase separation problems during the scrubbing operations. Since, as stated above, the higher (meth)acrylates cannot be purified by distillation, this polymer remains in the desired ester and influences the further processing and the quality of the polymers or copolymers prepared (U.S. Pat. No. 3,639,459, column 1, lines 40-55).

The use of polymerization inhibitors or inhibitor systems is generally recommended for substantially preventing the undesired polymer formation. In the case of the preparation of higher (meth)acrylates, for example, the following are used:

| | |
|---|---|
| DE 38 43 854: | α-Substituted phenolic compounds |
| DE-A 29 13 218: | Organic esters of phosphorous acid and |
| | monohydric or dihydric phenols |
| DE 38 43 938: | Unsubstituted phenol compounds and active carbon |
| EP 449 919: | Hydroquinones, sterically hindered |
| | hydroquinones, e.g. |
| | 2,5-di-tert-butylhydroquinone, sterically |
| | hindered phenols, e.g. tocopherols. |
| | Resulting coloration can be removed by means of |
| | alumina or active carbon. |
| DE-B 12 67 547: | Copper oxide/hydroquinone monomethyl ether |
| DE-A 20 03 579: | Phenothiazine |

Difficulties may occur in that the inhibitors or inhibitor systems on the one hand must prevent the free radical polymerization during the preparation and that, on the other hand, where they remain in the end product, they must not interfere with the use of the products, i.e. it must be possible to overcome their effect during use by triggering specific free radical reactions. It may therefore be necessary to separate off the preparation inhibitor in an expensive manner and to replace it by an application inhibitor (WO 91/08192).

U.S. Pat. No. 3,639,459 describes a process for the preparation of monomeric diesters of glycols and α,β-unsaturated acids in a solvent-free system with an excess of at least 10% of acid at a temperature below half the boiling point of the acid and purification by scrubbing with from 20 to 30% strength aqueous alkali metal hydroxide solution.

The disadvantage of the process described there is that the reaction times are about 20 hours, the water of esterification has to be removed by means of an azeotropic mixture with the acid under reduced pressure, necessitating complicated control of reduced pressure in the course of the reaction (regulation of 100 mm Hg to 5 mm Hg, depending on the progress of the reaction), and the reaction mixture is handled in a complicated manner, in some cases at below 0° C., in order to avoid ester cleavage by the highly concentrated alkali solution.

U.S. Pat. No. 4,187,383 describes a process for the esterification of (meth)acrylic acid with organic polyols at a reaction temperature of from 20 to 80° C. in the presence of from 50 to 5 000 ppm of an alkoxy-substituted phenol or alkylated alkoxyphenol as a polymerization inhibitor, by means of which products having a color number of 4.0 Gardner or less are obtained. In example 1 of said patent, the polyol and a solvent are initially taken, acrylic acid, polymerization inhibitor and catalyst are added during the heating phase, washing with 15% strength sodium hydroxide solution is effected after the reaction and the solvent is stripped. The reaction times are up to 35 hours, the Gardner color numbers are at best <1, mechanical stirring is required and a technically simpler circulation evaporator (see below) cannot be used. 1.0 Gardner corresponds here to about 160 APHA and 4.0 Gardner correspond to about 800 APHA, so that the color numbers achievable by this process are unsatisfactory.

For reducing the esterification time, EP-A 331 845 proposes initially taking a starting material in a stirred reactor, heating it to at least 100° C. and then continuously adding the other starting material within the period required for separating off at least 65% of the water of reaction and then heating until the reaction is complete. If required, a solvent which forms with water an azeotropic mixture having a boiling point above 100° C. can be added.

According to the examples, the solvent concentration in the reaction mixture is about 3.5% and the reaction temperature is 118-135° C.

According to this patent, temperatures below 100° C. considerably reduce the reaction rate.

The reaction mixture is said to be capable of being worked up by conventional methods. However, a suitable working-up procedure is not disclosed.

The disadvantages here are, inter alia, that strongly colored products are obtained, that the reaction mixture cannot be purified by scrubbing and that the end products have a high viscosity and a high salt content (cf. comparative examples).

Another disadvantage is that initially taking the total amount of alcohol during the heating-up in the presence of the acidic catalyst leads to increased ether formation (byproduct formation), but initially taking the total amount of (meth)acrylic acid leads to increased formation of Michael adducts and to a greater danger of polymerization, since the (meth)acrylic acid can then form Michael adducts and, in the worst case, can undergo uncontrolled polymerization (byproduct formation and safety problem).

In order to prevent a spontaneous polymerization, for example due to local overheating, thorough mixing must be ensured during the reaction, for which purpose EP-A 331 845 expressly provides a mechanical agitator (page 4, line 6). However, such mechanically moved parts are as far as possible avoided owing to their susceptibility to faults, and it is generally desirable to use circulation evaporators, in particular natural circulation evaporators, which are technically less susceptible to faults. Moreover, the reactor size is subject to limits because the specific wall area available for heat transfer decreases with increasing reactor size.

The process presented in EP-A 331 845 cannot, however, be carried out using a natural circulation evaporator when the highly viscous higher alcohols are initially taken, since the circulation is achieved only with difficulty, if at all, owing to the viscosity of the alcohols. However, reliable thorough mixing is absolutely essential owing to the safety problem described.

The addition of a solvent, as described in EP-A 331 845 or U.S. Pat. No. 4,187,383, is also not a solution here since the solvent frequently forms two phases with the higher alcohol before the beginning of the reaction, the mixing of which phases would be the object of the circulation evaporator, but the alcohol, being the component with the higher density, forms the lower phase so that here too circulation does not reliably occur.

In general, the reaction mixture obtained in the esterification substantially comprises the desired ester, the esterification catalyst, the inhibitors, the excess (meth)acrylic acid, any solvent and higher molecular weight byproducts (e.g. polymer, ether and Michael adducts).

The catalyst, the excess (meth)acrylic acid and, if required, parts of the inhibitors are as a rule separated off by treatment with aqueous bases, for example alkali solutions and/or salt solutions (DE-A 198 36 788) or solid pulverulent oxides, carbonates or hydroxides (DE-A 39 39 163, EP 449 919, EP 449 918, DE-A 1 493 004) or ion exchangers.

In these purification operations, the byproducts have an adverse effect in that they complicate or even prevent the phase separation in the individual scrubbing steps.

EP-A 890 568 therefore proposes a technically complicated centrifuge for separating the phases.

EP-A 933 353 proposes the addition of organic, polymeric, anionic water-soluble flocculants. The polymer formed is, however, generally tacky and prevents or complicates the filtration steps.

The excess (meth)acrylic acid can also be bound (rendered harmless) by treatment with epoxides, but the adducts formed remain in the end product (DE-C 38 36 370, DE-A 33 16 593).

Separating off any solvent present for removing the water of reaction is usually carried out by distillation.

Since some of the higher (meth)acrylates are used in the surface coating sector, the color plays an important role in addition to the purity and the viscosity.

Since the higher (meth)acrylates cannot be purified by distillation, various measures have been proposed for obtaining colorless end products.

DE-A 38 43 938 proposes the addition of active carbon during the esterification itself in order to prevent the formation of colored reaction products (column 2, lines 63-68). If discolorations nevertheless occur, an additional treatment with a suitable decolorizing agent, e.g. alumina, is recommended (column 5, lines 20-27).

EP-A 995 738 recommends carrying out the esterification in the presence of supercritical carbon dioxide in order to prevent, inter alia, discolorations.

The known processes have the disadvantage that they are technically complicated and/or require complicated apparatuses and/or additional assistants and are unsuitable on an industrial scale.

It is an object of the present invention to provide an economical process which permits the preparation of substantially colorless higher (meth)acrylates in high purity and high yield in a simple manner and without additional assistants on an industrial scale.

We have found that this object is achieved by a process for the preparation of higher (meth)acrylates by reacting (meth)acrylic acid and the alcohol in the presence of at least one acidic catalyst and at least one polymerization inhibitor and in the presence of a solvent which forms an azeotropic mixture with water and distilling off and condensing the azeotropic mixture, the condensate separating into an aqueous phase and an organic phase, wherein

a) the esterification is carried out in a reactor having a circulation evaporator and

b) in the presence of at least 10% by weight, based on the reaction mixture, of solvent, and either

c1) at least a part of the solvent, if required a part of the catalyst (mixture) and, if required, at least a part of the polymerization inhibitor (mixture) are initially taken in the esterification reactor and heated to the boiling point of the component having the lowest boiling point in the system and, as soon as the circulation is in operation, (meth)acrylic acid and alcohol and, if required, residual catalyst, polymerization inhibitor and solvent are metered in together or separately, or

c2) at least a part of the solvent, if required a part of the catalyst (mixture), if required at least a part of the polymerization inhibitor (mixture) and at least a part of the alcohol are initially taken in the esterification reactor and heated to the boiling point of the component having the lowest boiling point in the system and, as soon as the circulation is in operation, (meth)acrylic acid and, if required, the remaining alcohol and, if required, residual catalyst, polymerization inhibitor and solvent are metered in together or separately, or

c3) at least a part of the solvent, at least a part of the catalyst (mixture), at least a part of the polymerization inhibitor (mixture) and at least a part of the (meth)acrylic acid are initially taken in the esterification reactor and heated to the boiling point of the component having the lowest boiling point in the system and, as soon as the circulation is in operation, the alcohol and, if required, the remaining (meth)acrylic acid and, if required, residual catalyst, polymerization inhibitor and solvent are metered in together or separately, or

c4) at least a part of the solvent, at least a part of the catalyst (mixture), at least a part of the polymerization inhibitor (mixture), at least a part of the alcohol and a part of the (meth)acrylic acid are initially taken in the esterification reactor and heated to the boiling point of the component having the lowest boiling point in the system and, as soon as the circulation is in operation, if required the remaining alcohol and the remaining (meth)acrylic acid and, if required, residual catalyst, polymerization inhibitor and solvent are metered in together or separately.

Process variants c1), c2) and c4) are preferred, c1) and c2) are particularly preferred and cl) is very particularly preferred.

An advantageous embodiment comprises adding the catalyst only before the addition of the residual components, for example after the circulation is in operation and before the residual components are added.

This is advantageously effected if the boiling point of the initially taken reaction mixture is substantially reached, i.e. the heating is substantially complete. This is the case when the temperature of the mixture is not more than 20° C., preferably not more than 10° C., particularly preferably not more than 5° C., below the required boiling point.

The term (meth)acrylic acid is used here for acrylic acid and methacrylic acid.

The process found has the following advantages:

1. Only technically simple apparatuses are required

2. The novel process manages without assistants

3. There is no substantial polymer formation during esterification or working-up

4. There are no phase separation problems during the scrubbing operations

5. The end product is substantially colorless, i.e. the color number does not exceed 100 APHA

6. The end product is copper-free

7. A high degree of esterification is achieved

8. High yields are obtained

9. The end products have a low viscosity

The water which is formed in the esterification and forms an azeotropic mixture with the solvent is discharged via a column attached to the reactor and is condensed.

The condensate obtained (azeotropic mixture) separates into an aqueous phase, which is discharged and advantageously worked up (back-extraction of the acid contained), and a solvent phase, which is recycled as reflux into the column and, if required, partly into the reactor and/or evaporator, as described in DE-A 199 41 136 and the German Application having the application number 100 63 175.4.

A back-extraction of the (meth)acrylic acid contained is preferably effected with the solvent used as extracting agent, for example with cyclohexane, at from 10 to 40° C. and a ratio of aqueous phase to extracting agent of 1:5-30, preferably 1:10-20. The acid contained in the extracting agent can preferably be fed directly into the esterification.

After the end of the esterification, the hot reaction mixture is rapidly cooled, if required diluted with solvent, prewashed, neutralized and, if required, subsequently washed.

The solvent is then separated from the desired ester by distillation, the main amount of the solvent being separated off by distillation in a first step and the remainder of the solvent then being removed by stripping with a gas which is inert under the reaction conditions, preferably an oxygen-containing gas, particularly preferably air or air/nitrogen mixtures.

Advantageously, the stripping gas is heated.

Suitable higher alcohols in addition to high-boiling monohydric alcohols of 10 carbon atoms or more, preferably of 10 to 30, particularly preferably 10 to 20, carbon atoms, are also diols and polyols having 2 to 10, preferably 2 to 6, hydroxyl groups.

Examples of high-boiling monohydric alcohols are tert-butylcyclohexanol, lauryl alcohol (1-dodecanol), myristyl alcohol (1-tetradecanol), cetyl alcohol (1-hexadecanol), stearyl alcohol (1-octadecanol), 9-cis-octadecen-1-ol (oleyl alcohol), 9-trans-octadecen-1-ol (elaidyl alcohol), 9-cis-octadecen-1,12-diol (ricinoleyl alcohol), all-cis-9,12-octadecadien-1-ol (iinoleyl alcohol), all-cis-9,12,15-octadecatrien-1-ol (linolenyl alcohol), 1-eicosanol (arachidyl alcohol), 9-cis-eicosen-1-ol (gadoleyl alcohol), 1-docosanol (behenyl alcohol), 13-cis-docosen-1-ol (erucyl alcohol) and 1,3-trans-docosen-1-ol (brassidyl alcohol).

Examples of diols and polyols are 1,4-butanediol, neopentylglycol, 1,6-hexanediol, glycerol, trimethylolpropane, ditrimethylolpropane, trimethylolethane, pentaerythritol, dipentaerythritol, 1,2-, 1,3- or 1,4-bis(hydroxymethyl)cyclohexane, bisphenol A, bisphenol F or 2,2-bis(4-hydroxycyclohexyl)propane. Among these, 1,4-butanediol, neopentylglycol, 1,6-hexanediol, glycerol, trimethylolpropane, ditrimethylolpropane, trimethylolethane, pentaerythritol and dipentaerythritol are preferred.

Also suitable are polyetherols and polyesterols having one or more hydroxyl groups, preferably having an average OH functionality of from 1 to 10, preferably from 1 to 5, particularly preferably from 1 to 3, for example ethoxylated and/or propoxylated monohydric and polyhydric alcohols, phenols or fatty amines.

Preferred polyetherols and polyesterols are those having a molar mass of less than 2 000, preferably from 100 to 2 000, particularly preferably from 100 to 1 000, very particularly preferably from 100 to 400, g/mol.

Examples of these are

polytetrahydrofuran having a molar mass of from 162 to 2 000, poly-1,3-propanediol having a molar mass of from 134 to 1 178, polypropylene glycol having a molar mass of from 134 to 1 178, preferably di- and tripropylene glycol,

polyethylene glycol having a molar mass of from 106 to 898, polyethylene glycol methyl ether having a molar mass of from 120 to 912, preferably diethylene glycol monomethyl ether and triethylene glycol monomethyl ether,

polyethylene glycol ethyl ether having a molar mass of from 134 to 926, preferably diethylene glycol monoethyl ether and triethylene glycol monoethyl ether,

Lutensol®, Pluriol®, Pluriol E® or Pluriol P® grades from BASF AG.

They may furthermore be ethoxylated and/or propoxylated alcohols and mixed ethoxylated/propoxylated alcohols, such as

R¹—(O—CH₂—CH₂)ₓ—OH or

R¹—(O—CH(CH₃)—CH₂)—OH or R¹—(O—CH₂—CH(CH₃))ₓ—OH,

where

R¹ is C₁- to C₂₂-alkyl and

x is an integer from 1 to 20.

Examples of R¹ are methyl, ethyl, isopropyl, n-propyl, allyl, n-butyl, isobutyl, sec-butyl, tert-butyl, n-hexyl, n-heptyl, n-octyl, n-decyl, n-dodecyl, n-tetradecyl, n-hexadecyl, n-octadecyl or n-eicosyl.

Those alcohols which are mono- to pentadecaethoxylated and/or -propoxylated, particularly preferably mono- to decaethoxylated and/or -propoxylated, very particularly preferably di- to pentaethoxylated and/or -propoxylated, per hydroxyl group are preferably used.

Examples of alcohols which can be used after ethoxylation and/or propoxylation are methanol, ethanol, isopropanol, n-propanol, allyl alcohol, n-butanol, isobutanol, sec-butanol, tert-butanol, 2,2-dimethyl-1,2-ethanediol, 1,3-propanediol, 1,2-butanediol, 1,4-butanediol, 1,4-butynediol, 1,4-butenediol, n-hexanol, n-heptanol, n-octanol, n-decanol, n-dodecanol (lauryl alcohol), 2-ethylhexanol, 3-methylpentane-1,5-diol, 2-ethylhexane-1,3-diol, 2,4-diethyloctane-1,3-diol, 1,6-hexanediol, cyclopentanol, cyclohexanol, cyclooctanol, cyclododecanol, but-2-yne-1,4-diol, trimethylolpropane, trimethylolethane, neopentyl glycol, pentaerythritol, 2-methyl-1,3-propanediol, glycerol, ditrimethylolpropane, dipentaerythritol, sugar alcohols, bisphenol A, bisphenol F, bisphenol B, bisphenol S, 2,2-bis(4-hydroxycyclohexyl)propane, 1,1-, 1,2-, 1,3- and 1,4-cyclohexanedimethanol, 1,2-, 1,3- or 1,4-cyclohexanediol, lauryl alcohol (1-dodecanol), myristyl alcohol (1-tetradecanol), cetyl alcohol (1-hexadecanol), stearyl alcohol (1-octadecanol), 9-cis-octadecen-1-ol (oleyl alcohol), 9-trans-octadecen-1-ol (elaidyl alcohol), 9-cis-octadecene-1,12-diol (ricinoleyl alcohol), all-cis-9,12-octadecadiene-1-ol (linoleyl alcohol), all-cis-9,12,15-octadecatrien-1-ol (linolenyl alcohol), 1-eicosanol (arachidyl alcohol), 9-cis-eicosen-1-ol, (gadoleyl alcohol), 1-docosanol (behenyl alcohol), 13-cis-docosen-1-ol (erucyl alcohol) and 1,3-trans-docosen-1-ol (brassidyl alcohol).

Ethoxylated and/or propoxylated methanol, trimethylolpropane, pentaerythritol, dipentaerythritol, glycerol, ethylene glycol, 1,6-hexanediol, 1,4-butanediol, 1,4-butynediol, propylene glycol and neopentyl glycol are particularly preferred.

Furthermore, ethoxylated and/or propoxylated alkylphenols, e.g. nonylphenol, can be used.

The novel process substantially comprises the following stages:

1. Esterification

The esterification apparatus consists of a reactor having a circulation evaporator and an attached distillation column with condenser and phase separation vessel.

The reactor may be, for example, a reactor having double-jacket heating and/or internal heating coils. A reactor having an external heat exchanger and natural or forced circulation (using a pump), particularly preferably natural circulation, in which the circulation stream is effected without mechanical aids, is preferably used.

Suitable circulation evaporators are known to a person skilled in the art and are described, for example, in R. Billet, Verdampfertechnik, HTB-Verlag, Bibliographisches Institut Mannheim, 1965, 53. Examples of circulation evaporators are tube-bundle heat exchangers, plate-type heat exchangers, etc.

Of course, a plurality of heat exchangers may also be present in the circulation.

The distillation column is of a design known per se and has the conventional internals. Suitable column internals are in principle all conventional internals, for example trays, stacked packings and/or dumped packings. Among the trays, bubble trays, sieve trays, valve trays, Thormann trays and/or dual-flow trays are preferred; among the dumped packings, those having rings, coils, saddles or braids are preferred.

As a rule, from 5 to 20 theoretical plates are sufficient.

The condenser and the separation vessel are of conventional design.

(Meth)acrylic acid and the alcohols are used as a rule in equivalent amounts, based on the hydroxyl groups of the alcohol, but it is also possible to use less than the stoichiometric amount or an excess of (meth)acrylic acid.

Preferably, an excess of (meth)acrylic acid of 5-100, preferably from 5 to 50, particularly preferably from 5 to 25, in particular from 5 to 10, mol % is established per hydroxyl group (equivalent) to be esterified.

Suitable esterification catalysts are the conventional mineral acids and sulfonic acids, preferably sulfuric acid, phosphoric acid, alkanesulfonic acids (e.g. methanesulfonic acid, trifluoromethanesulfonic acid) and arylsulfonic acids (e.g. benzene-, p-toluene- or dodecylbenzenesulfonic acid) or mixtures thereof, but acidic ion exchangers are also possible.

Sulfuric acid, methanesulfonic acid and p-toluenesulfonic acid and mixtures thereof are particularly preferred.

They are used as a rule in an amount of 0.1-5, preferably 0.5-5, particularly preferably 1-4, very particularly preferably 2-4, % by weight, based on the esterification mixture.

If required, the esterification catalyst can be removed from the reaction mixture with the aid of an ion exchanger. The ion exchanger may be added directly to the reaction mixture and then filtered off, or the reaction mixture can be passed over an ion exchanger bed.

Preferably, the esterification catalyst is left in the reaction mixture and is removed by washing (see below).

Suitable polymerization inhibitors which can be used in the esterification are phenothiazine, monohydric and polyhydric phenols, which may have one or more alkyl groups, e.g. alkylphenols, for example o-, m- or p-cresol (methylphenol), 2-tert-butyl-4-methylphenol, 6-tert-butyl-2,4-dimethylphenol, 2,6-di-tert-butyl-4-methylphenol, 2-methylhydroquinone, 2,5-di-tert-butylhydroquinone, 2-tert-butylphenol, 4-tert-butylphenol, 2,4-di-tert-butylphenol, 2-methyl-4-tert-butylphenol, 4-tert-butyl-2,6-dimethylphenol, 2,5-di-tert-butylhydroquinone, toluhydroquinone or 2,2′-methylenebis(6-tert-butyl-4-methylphenol), hydroxyphenols, for example hydroquinone, pyrocatechol (1,2-dihydroxybenzene) or benzoquinone, aminophenols, e.g. para-aminophenol, nitrosophenols, e.g. para-nitrosophenol, alkoxyphenols, for example 2-methoxyphenol (guajacol, pyrocatechol monomethyl ether), 2-ethoxyphenol, 2-isopropoxyphenol, 4-methoxyphenol (hydroquinone monomethyl ether), mono- or di-tert-butyl-4-methoxyphenol, tocopherols, e.g. α-tocopherol and 2,3-dihydro-2,2-dimethyl-7-hydroxybenzofuran (2,2-dimethyl-7-hydroxycoumarane), phosphorus compounds, e.g. triphenyl phosphite, hypophosphorous acid or alkyl esters of phosphorous acid, copper or manganese, cerium, nickel, chromium or copper salts, for example chlorides, sulfates, salicylates, tosylates, acrylates or acetates thereof, 4-hydroxy-2,2,6,6-tetramethylpiperidin-N-oxyl, 4-oxo-2,2,6,6-tetramethylpiperidin-N-oxyl, 4-acetoxy-2,2,6,6-tetramethylpiperidin-N-oxyl, 2,2,6,6-tetramethylpiperidin-N-oxyl, 4,4′,4″-tris(2,2,6,6-tetramethylpiperidin-N-oxyl) phosphite or 3-oxo-2,2,5,5-tetramethylpyrrolidin-N-oxyl, N,N-diphenylamine, N-nitrosodiphenylamine, N,N′-dialkyl-para-phenylenediamines and mixtures thereof.

At least one compound from the group consisting of hydroquinone, hydroquinone monomethyl ether, 2-tert-butyl-4-methylphenol, 6-tert-butyl-2,4-dimethylphenol, 2,6-di-tert-butyl-4-methylphenol, 2-tert-butylphenol, 4-tert-butylphenol, 2,4-di-tert-butylphenol, 2-methyl-4-tert-butylphenol, 4-tert-butyl-2,6-dimethylphenol, hypophosphorous acid, copper acetate, copper chloride and copper salicylate is preferably used as the polymerization inhibitor (mixture).

The polymerization inhibitor (mixture) is preferably used in the form of an aqueous solution.

To further support the stabilization, an oxygen-containing gas, preferably air or a mixture of air and nitrogen (air having a low oxygen content) may be present.

This oxygen-containing gas is preferably metered into the bottom region of a column and/or into a circulation evaporator.

The polymerization inhibitor (mixture) is used in a total amount of 0.01-1, preferably 0.02-0.8, particularly preferably 0.05-0.5, % by weight, based on the esterification mixture.

Suitable solvents for the azeotropic removal of the water of reaction are in particular aliphatic, cycloaliphatic and aromatic hydrocarbons or mixtures thereof.

n-Pentane, n-hexane, n-heptane, cyclohexane, methylcyclohexane, benzene, toluene or xylene are preferably used. Cyclohexane, methylcyclohexane and toluene are particularly preferred.

The amount used is 10-200, preferably 20-100, particularly preferably 30-100, % by weight, based on the sum of alcohol and (meth)acrylic acid.

The reaction temperature is in general 60-140° C., preferably 70-110° C., very particularly preferably 75-100° C. The initial temperature is in general less than 100° C., preferably less than 90° C., particularly preferably less than 80° C. As a rule, the final temperature of the esterification is 5-30° C. higher than the initial temperature. The temperature of the esterification can be determined and controlled by varying the solvent concentration in the reaction mixture, as described in DE-A 199 41 136 and the German Application having the application number 100 63 175.4.

The esterification can be carried out at atmospheric, superatmospheric or reduced pressure, atmospheric pressure preferably being employed.

The reaction time is as a rule 3-20, preferably 5-15, particularly preferably from 7 to 12, hours.

A typical procedure for process variant cl) comprises initially taking a mixture of at least a part of the solvent, if required a part of the catalyst (mixture) and, if required, at least a part of the inhibitor (mixture) in the reactor and heating them to the boiling point. Here, boiling point means the temperature of the component having the lowest boiling point in the system.

Preferably from 50 to 100, particularly preferably 75-100, in particular 100, % of the solvent are initially taken. Preferably from 0 to 100, particularly preferably 0-75, in particular 0-50, % of the polymerization inhibitor (mixture) are initially taken.

Preferably from 0 to 50, particularly preferably from 0 to 25, in particular 0, % of the catalyst (mixture) are initially taken. Accordingly, 50-100%, 75-100% or 100%, respectively, of the catalyst are not added until after the heating is substantially complete and before further substances, in particular alcohol and/or acid, are added.

In the case of an esterification reactor having natural circulation, particular attention is paid to ensuring circulation of the mixture. This is established, for example, by means of inspection windows or instrumentation known per se, for example for flow measurement in the circulation.

As soon as the circulation is in operation, the (meth)acrylic acid and the alcohol and, if required, the remaining catalyst (mixture), polymerization inhibitor (mixture) and solvent can be metered in together or separately.

A typical procedure for process variant c2) comprises initially taking a mixture of at least a part of the alcohol, at least a part of the solvent, if required a part of the catalyst (mixture) and, if required, at least a part of the inhibitor (mixture) in the reactor and heating them to the boiling point.

Preferably from 25 to 100, particularly preferably 50-100, very particularly preferably 75-100, in particular 100, % of the alcohol are initially taken.

Preferably from 50 to 100, particularly preferably 75-100, in particular 100, % of the solvent are initially taken.

Preferably from 0 to 100, particularly preferably 0-75, in particular 0-50, % of the polymerization inhibitor (mixture) are initially taken.

Preferably from 0 to 50, particularly preferably from 0 to 25, in particular 0, % of the catalyst (mixture) are initially taken. Accordingly, 50-100%, 75-100% or 100%, respectively, of the catalyst are not added until after the heating is substantially complete and before further substances, in particular alcohol and/or acid, are added.

In the case of an esterification reactor having natural circulation, particular attention is paid to ensuring the circulation of the mixture (cf. process variant c1)).

As soon as the circulation is in operation, the (meth)acrylic acid and, if required, residual alcohol, residual catalyst (mixture), polymerization inhibitor (mixture) and solvent can be metered in together or separately.

A typical procedure for process variant c3) comprises initially taking the mixture of at least a part of the (meth)acrylic acid, at least a part of the solvent, if required a part of the catalyst (mixture) and at least a part of the inhibitor (mixture) in the reactor and heating them to the boiling point.

Preferably from 25 to 100, particularly preferably 50-100, very particularly preferably 75-100, in particular 100, % of the (meth)acrylic acid are initially taken.

Preferably from 50 to 100, particularly preferably 75-100, in particular 100, % of the solvent are initially taken.

Preferably from 50 to 100, particularly preferably 75-100, in particular 100, % of the polymerization inhibitor (mixture) are initially taken.

Preferably from 0 to 50, particularly preferably from 0 to 25, in particular 0, % of the catalyst (mixture) are initially taken. Accordingly, 50-100%, 75-100% or 100%, respectively, of the catalyst are not added until after the heating is substantially complete and before further substances, in particular alcohol and/or acid, are added.

In the case of an esterification reactor having natural circulation, particular attention is paid to ensuring the circulation of the mixture (cf. process variant c1)).

As soon as the circulation is in operation, the alcohol and, if required, the residual (meth)acrylic acid, residual catalyst (mixture), polymerization inhibitor (mixture) and solvent can be metered in together or separately.

A typical procedure for process variant c4) comprises initially taking a mixture of a part of the (meth)acrylic acid, at least a part of the alcohol, at least a part of the solvent, if required a part of the catalyst (mixture) and at least a part of the inhibitor (mixture) in the reactor and heating them to the boiling point.

Preferably from 5 to 90, particularly preferably 10-80, very particularly preferably 20-75, % of the (meth)acrylic acid are initially taken.

Preferably from 10 to 100, particularly preferably 25-100, very particularly preferably 50-90, % of the alcohol are initially taken.

Preferably from 50 to 100, particularly preferably 75-100, in particular 100, % of the solvent are initially taken.

Preferably from 50 to 100, particularly preferably 75-100, in particular 100, % of the polymerization inhibitor (mixture) are initially taken.

Preferably from 0 to 50, particularly preferably from 0 to 25, in particular 0, % of the catalyst (mixture) are initially taken. Accordingly, 50-100%, 75-100% or 100%, respectively, of the catalyst are not added until after the heating is substantially complete and before further substances, in particular alcohol and/or acid, are added.

In the case of an esterification reactor having natural circulation, particular attention is paid to ensuring the circulation of the mixture (cf. process variant c1)).

As soon as the circulation is in operation, any residual alcohol and the residual (meth)acrylic acid, residual catalyst (mixture), polymerization inhibitor (mixture) and solvent can be metered in together or separately.

Here, together or separately means via common or separate lines, and the metering can be effected in general with ratio control.

The metering is effected as a rule in the course of 0.5-5 hours, continuously or a little at a time.

The (meth)acrylic acid which can be used is not limited and, in the case of crude (meth)acrylic acid, may contain, for example, the following components:

| | |
|---|---|
| (Meth)acrylic acid | 90-99.9% by weight |
| Acetic acid | 0.05-3% by weight |
| Propionic acid | 0.01-1% by weight |
| Diacrylic acid | 0.01-5% by weight |
| Water | 0.05-5% by weight |
| Aldehydes | 0.01-0.3% by weight |
| Inhibitors | 0.01-0.1% by weight |
| Maleic acid | 0.001-0.5% by weight |
| (anhydride) | |

The crude (meth)acrylic acid used is as a rule stabilized with 200-600 ppm of phenothiazine or other stabilizers in amounts which permit comparable stabilization.

It is of course also possible to use pure (meth)acrylic acid having, for example, the following purity:

| | |
|---|---|
| (Meth) acrylic acid | 99.7-99.99% by weight |
| Acetic acid | 50-1000 ppm by weight |
| Propionic acid | 10-500 ppm by weight |
| Diacrylic acid | 10-500 ppm by weight |
| Water | 50-1000 ppm by weight |
| Aldehydes | 1-500 ppm by weight |
| Inhibitors | 1-300 ppm by weight |
| Maleic acid (anhydride) | 1-200 ppm by weight |

The pure (meth)acrylic acid used is as a rule stabilized with 100-300 ppm of hydroquinone monomethyl ether or other storage stabilizers in amounts which permit comparable stabilization.

The preferably used natural circulation can be supported by metering a solvent, preferably the solvent which originates from the organic phase of the column attached to the reactor, as described in the German Application having the application number 100 63 175.4, into the heat exchanger.

The water formed in the reaction is removed continuously from the reaction mixture, as an azeotropic mixture with the solvent, via the column attached to the reactor, and is condensed, the condensate separating into an aqueous phase and an organic phase.

The aqueous phase of the condensate, which as a rule contains 0.1-10% by weight of (meth)acrylic acid, is separated off and discharged. Advantageously, the (meth)acrylic acid contained therein can be extracted with an extracting agent, for example with cyclohexane, at from 10 to 40° C. and a ratio of aqueous phase to extracting agent of 1:5-30, preferably 1:10-20, and can be recycled into the esterification.

Some or all of the organic phase can be recycled as reflux into the column and any excess remainder can be recycled into the reactor. When a natural circulation is used, a part of this phase can, if required, be introduced into the heat exchanger in the reactor circulation in order to support the natural circulation, preferably at least 10, particularly preferably at least 15, very particularly preferably at least 20, % by weight of the organic phase.

An advantageous variant comprises passing the organic phase (solvent phase) into a storage container and removing from this container the amount of solvent required in each case for maintaining the reflux, for passing into the circulation evaporator and as solvent for reaction and extraction.

To further support the circulation, an inert gas, preferably an oxygen-containing gas, particularly preferably air or a mixture of air and nitrogen (air having a low oxygen content) can be passed into the circulation, for example in amounts of 0.1-1, preferably 0.2-0.8, particularly preferably 0.3-0.7, m³/m³h, based on the volume of the reaction mixture.

The course of the esterification can be monitored by monitoring the amount of water discharged and/or the decrease in the (meth)acrylic acid concentration in the reactor.

The reaction can be stopped, for example, as soon as 90, preferably at least 95, particularly preferably at least 98, % of the theoretically expected amount of water has been discharged by means of the solvent.

After the end of the esterification, the reactor mixture is rapidly cooled in a conventional manner to a temperature from 10 to 30° C., if required a concentration of desired ester of 60-80% is established by adding solvent and the mixture is fed to a scrubbing apparatus.

2. Preliminary Wash

The reaction mixture from 1. is treated in a scrubbing apparatus with water or a 5-30, preferably 5-20, particularly preferably 5-15, % strength by weight sodium chloride, potassium chloride, ammonium chloride, sodium sulfate or ammonium sulfate solution, preferably sodium chloride solution.

The ratio of reaction mixture to wash liquid is as a rule 1 0.1-1, preferably 1:0.2-0.8, particularly preferably 1:0.3-0.7.

The washing can be carried out, for example, in a stirred container or in other conventional apparatuses, for example in a column or mixer-settler apparatus.

All extraction and scrubbing methods and apparatuses known per se can be used for washing in the novel process, for example those which are described in Ullmann's Encyclopedia of Industrial Chemistry, 6th ed, 1999 Electronic Release, Chapter: Liquid—Liquid Extraction—Apparatus. For example, these may be one-stage or multistage, preferably one-stage, extractions and those effected by the cocurrent or countercurrent procedure, preferably the countercurrent procedure.

Sieve tray columns or columns containing stacked or dumped packings, stirred containers or mixer-settler apparatuses and columns having rotating internals are preferably used.

The preliminary washing is preferably used when metal salts, preferably copper or copper salts, are (concomitantly) used as inhibitors.

3. Neutralization

The organic phase of the preliminary wash, which may still contain small amounts of catalyst and the main amount of excess (meth)acrylic acid, is neutralized with a 5-25, preferably 5-20, particularly preferably 5-15, % strength by weight aqueous solution of a base, e.g. sodium hydroxide solution, potassium hydroxide solution, sodium bicarbonate, sodium carbonate, potassium bicarbonate, calcium hydroxide, ammonia water or potassium carbonate, to which, if required, 5-15% by weight of sodium chloride, potassium chloride, ammonium chloride or ammonium sulfate may have been added, preferably with sodium hydroxide solution or sodium hydroxide/sodium chloride solution.

The base is added in a manner such that the temperature of the apparatus does not increase above 35° C., and is preferably from 20 to 35° C. and the pH is 10-14. The removal of the heat of neutralization is preferably effected by cooling the container with the aid of internal cooling coils or via double-jacket cooling.

The ratio of reaction mixture to neutralizing liquid is as a rule 1:0.1-1, preferably 1:0.2-0.8, particularly preferably 1:0.3-0.7.

Regarding the apparatus, the statements made under “2.” are applicable.

4. Subsequent Washing (Optional)

In order to remove traces of base or salt from the neutralized reaction mixture, it may be advantageous to carry out subsequent washing with water or a 5-30, preferably 5-20, particularly preferably 5-15, % strength by weight sodium chloride, potassium chloride, ammonium chloride or ammonium sulfate solution, preferably water or sodium chloride solution, analogously to the preliminary washing.

The ratio of reaction mixture to wash liquid is as a rule 1:0.1-1, preferably 1:0.2-0.8, particularly preferably 1:0.3-0.7.

Regarding the apparatus, the statements made under “2.” are applicable.

5. Solvent Distillation

The washed reaction mixture is mixed with an amount of storage stabilizer, preferably hydroquinone monomethyl ether, such that, after removal of the solvent, 100-500, preferably 200-500, particularly preferably 200-400, ppm thereof are contained in the desired ester (residue).

The removal of the main amount of solvent by distillation is effected, for example, in a stirred kettle having double-jacket heating and/or internal heating coils under reduced pressure, for example at 20-700, preferably from 30 to 500, particularly preferably 50-150, mbar and 40-80° C.

Of course, the distillation can also be effected in a falling-film or thin-film evaporator. For this purpose, the reaction mixture is passed through the apparatus, preferably several times by circulation, under reduced pressure, for example at 20-700, preferably from 30 to 500, particularly preferably 50-150, mbar and 40-80° C.

An inert gas, preferably an oxygen-containing gas, particularly preferably air or a mixture of air and nitrogen (air having a low oxygen content) can advantageously be passed into the distillation apparatus, for example 0.1-1, preferably 0.2-0.8, particularly preferably 0.3-0.7, m³/m³h, based on the volume of the reaction mixture.

The solvent content of the residue after the distillation is as a rule less than 5, preferably 0.5-5, particularly preferably from 1 to 3, % by weight.

The solvent separated off is condensed and preferably reused.

6. Solvent Stripping

The desired ester, which still contains small amounts of solvent, is heated to 50-80° C. and the residual amounts of solvent are removed by means of suitable gas in a suitable apparatus.

Suitable apparatuses are, for example, columns of a design known per se, which have the conventional internals, for example trays, dumped packings or stacked packings, preferably dumped packings. Suitable column internals are in principle all conventional internals, for example trays, stacked packings and/or dumped packings. Among the trays, bubble trays, sieve trays, valve trays, Thormann trays and/or dual-flow trays are preferred; among the dumped packings, those comprising rings, coils, saddles, Raschig, Intos or Pall rings, barrel or Intalox saddles, Top-Pak, etc. or braids are preferred.

A falling-film, thin-film or wiped-film evaporator, for example a Luwa, Rotafilm or Sambay evaporator, which is equipped, for example, with a demister as spray protection, is also possible here.

Suitable gases are gases which are inert under the stripping conditions, preferably oxygen-containing gases, particularly preferably air or mixtures of air and nitrogen (air having a low oxygen content), in particular those which are preheated to from 50 to 100° C.

The amount of stripping gas is, for example, 5-20, particularly preferably 10-20, very particularly preferably from 10 to 15, m³/m³h, based on the volume of the reaction mixture.

If necessary, the ester may be subjected to a filtration after the stripping, in order to remove precipitated traces of salts.

The higher (meth)acrylates obtained by the novel process are clear and substantially colorless (color number <100 APHA corresponds to Hazen) and contain, as a rule, not more than 0.05% by weight of (meth)acrylic acid, not more than 0.1% by weight of solvent, not more than 0.1 ppm of copper and not more than 20 ppm of sodium.

The present invention furthermore relates to a reaction mixture which contains substantially trimethylolpropane triacrylate and is obtainable by process variant c1).

For this purpose, at least 10, preferably at least 20, particularly preferably at least 30, % by weight, based on the sum of trimethylolpropane and acrylic acid, of the solvent and at least a part of the polymerization inhibitor (mixture) are initially taken in a reactor having a circulation, preferably a natural circulation, and heated, and trimethylolpropane and acrylic acid are metered in in a ratio of 1:3.3-4.5, preferably 1:3.6-4.5, particularly preferably 1:3.9-4.5, the acidic catalyst required for the reaction, preferably para-toluenesulfonic acid, being added before trimethylolpropane and acrylic acid are metered in.

The reaction temperature is brought to 70-110° C., preferably 75-100° C. The initial temperature is in general less than 100° C., preferably less than 90° C., particularly preferably less than 80° C. As a rule, the final temperature of the esterification is 5-30° C. higher than the initial temperature.

The duration of the reaction is less than 20, preferably less than 15, particularly preferably less than 10, hours.

The reaction mixture is then, if required, prewashed, neutralized and, if required, subsequently washed, and the solvent is then removed by distillation and stripping to a content of less than 0.5, preferably less than 0.3, % by weight.

Trimethylolpropane triacrylate is of course also obtainable by variant c2), c3) or c4), but variant c1) is preferred and the reaction procedure described is particularly preferred.

The reaction mixture thus obtainable and containing substantially trimethylolpropane triacrylate has a color number of less than 100 APHA, preferably less than 80 APHA, particularly preferably less than 60 APHA, and a viscosity (according to DIN 51562 at 25° C.) of less than 160, preferably less than 140, particularly preferably less than 120, mPa.s and an ester number (according to DIN 53401) of from 480 to 570, preferably from 500 to 570, particularly preferably from 520 to 560.

A reaction mixture containing substantially acrylates of alkoxylated trimethylolpropane is also obtainable analogously to this method. For this purpose, an alkoxylated trimethylolpropane which is obtainable by reacting trimethylolpropane with alkylene oxides is used as feedstock in the esterification. This alkoxylation is not essential to the invention and is known per se to a person skilled in the art. Suitable alkylene oxides are, for example, ethylene oxide, propylene oxide, isobutylene oxide, vinyloxirane and/or styrene oxide, preferably ethylene oxide, propylene oxide and/or isobutylene oxide, particularly preferably ethylene oxide and/or propylene oxide.

In particular, reaction mixtures containing substantially acrylates of alkoxylated trimethylolpropane which are obtainable by reacting from 0.5 to 10 mol of ethylene oxide and/or propylene oxide per mol of trimethylolpropane are preferred. The viscosities and color numbers of the reaction mixtures obtainable differ depending on the alkoxylated trimethylolpropane used.

In addition to being used for the preparation of methacrylates, the novel process can also be used for the preparation of esters of other α,β-ethylenically unsaturated carboxylic acids, such as crotonic acid, itaconic acid, maleic acid, fumaric acid or citraconic acid, with alcohols and in particular the abovementioned alcohols.

ppm and percentage data used in this document are by weight, unless stated otherwise.

APHA color numbers were determined according to DIN-ISO 6271.

Viscosities were determined according to DIN 51562 at 25° C., unless stated otherwise.

### EXAMPLE 1

3 g of hydroquinone monomethyl ether in the form of a 2% strength aqueous solution, 6 g of 50% strength hypophosphorous acid, 1 g of copper(II) chloride in the form of a 40% strength aqueous solution and 2 000 g of cyclohexane were initially taken in a 10 l reactor having an external natural circulation evaporator, distillation column, condenser and phase separation vessel and were refluxed. The temperature in the reactor was from about 70 to 75° C. The natural circulation evaporator was a tube-bundle heat exchanger heated by means of heat transfer oil. In addition, air was passed into the natural circulation evaporator, the amount of air being 2 l/h. The column had a diameter of 50 mm and a length of 700 mm and was filled with 8 mm glass rings. After the initially taken mixture circulated in the evaporator, 300 g of 65% strength p-toluenesulfonic acid were added and then 2 144 g of trimethylolpropane and 3 810 g of acrylic acid were metered in.

The azeotropic mixture separated off via the column was condensed and the aqueous phase was discharged. The cyclohexane phase was partly (800 g/h) metered into the natural circulation evaporator from below and partly fed as reflux to the distillation column and the internal temperature of the reactor was thus controlled.

The minimum reflux of the distillation column was 1 600 g/h. After the end of the addition (2 hours), the reaction temperature was increased to 95-99° C. in the course of 2 hours. After an esterification time of 10 hours, the experiment was terminated and the reaction mixture was rapidly cooled to 20° C.

The aqueous phase separated off (98% of theory) contained 4.7% of acrylic acid. The reaction mixture (6 960 g) contained 4.1% of acrylic acid.

By adding cyclohexane to the reaction mixture, a crude ester concentration of about 65% was established and washing was effected at 20° C. with an 8% strength sodium chloride solution (1 500 g) in a stirred container. After the aqueous phase had been separated off, a 12% strength sodium hydroxide solution was added to the organic phase until a pH of 13 was obtained, the temperature being kept at 20-40° C. After the aqueous phase had been separated off, the neutralized organic phase was subsequently washed with a 12% strength sodium chloride solution (1 500 g).

The washed organic phase was mixed with 1 g of hydroquinone monomethyl ether and heated to 60° C. in a stirred container and the cyclohexane was separated off by distillation (final reduced 25 pressure 100 mbar). The residual cyclohexane content was about 1.5%.

In a packed column (5 mm Raschig rings), the residual cyclohexane was reduced to a content of <0.1% at 80° C. by means of preheated air (75° C., 30 l/h) and then filtered.

The end product (4 550 g) was clear and substantially colorless (Hazen color number 30). The degree of esterification determined by means of the ester number was 90% of theory, the yield was 96% of theory, the acrylic acid content was 0.04%, and the cyclohexane content was 0.03%, the sodium content was 12 ppm, the copper content was <0.1 ppm and the viscosity was 110 mPa.s.

### EXAMPLE 2

### COMPARATIVE EXAMPLE

The procedure was as in example 1, except that all starting materials were initially taken together in the reactor from the outset. After an esterification time of 8 hours, the mixture was cooled to 20° C., prewashed with sodium chloride solution and then neutralized (pH 13). No phase separation could be achieved.

### EXAMPLE 3

### COMPARATIVE EXAMPLE

The procedure was as in example 1, except that a reaction temperature of 120° C. was established by reducing the amount of cyclohexane to 500 g, which reaction temperature increased to 130° C. toward the end of the reaction. After a reaction time of 6 hours, the mixture was cooled and was washed as usual. The phase separation time in the neutralization step was three times longer than in example 1.

The color number was 100 APHA and the viscosity was 170 mPa.s.

### EXAMPLE 4

### COMPARATIVE EXAMPLE

According to EP 331 845 A2, example 2, a mixture of 711 g of acrylic acid, 6 g of methanesulfonic acid, 40 g of toluene and 1.2 g of hydroquinone was heated to 120° C. in a stirred reactor having double-jacket heating and an attached column with condenser and separation vessel, and a trimethylolpropane (402 g) heated to 145° C. was metered in over 2.5 hours. The removal of the water was started about 50 minutes after the beginning of the addition of trimethylolpropane. After a reaction time of 5 hours, 95% of the theoretical amount of water had been discharged.

The strongly colored reaction mixture (APHA color number: 500) was neutralized with a 12% strength sodium hydroxide solution at 25-40° C. (pH 13). No phase separation occurred even after standing for several hours.

### EXAMPLE 5

### COMPARATIVE EXAMPLE

The esterification was carried out as in example 4. The reaction mixture cooled to 25° C. was treated with sodium carbonate powder and filtered. The neutralized reaction mixture had to be filtered twice in order to obtain a clear product (color number 400). A treatment with active carbon (50 g) led only to an insignificant improvement in the color number (300 APHA). After removal of the toluene under reduced pressure (80° C., 20 mbar), the viscosity was 180 mPa.s and the sodium content 60 ppm.

### EXAMPLE 6

3 264 g of tripropylene glycol, 3 g of methoxyphenol in the form of a 2% strength aqueous solution, 6 g of 50% strength hypophosphorous acid, 1 g of copper(II) chloride in the form of a 20% strength aqueous solution, 300 g of 65% strength p-toluenesulfonic acid and 1 800 g of cyclohexane were initially taken in a 10 l reactor having an external natural circulation evaporator, distillation column, condenser and phase separation vessel.

The natural circulation evaporator was a tube-bundle heat exchanger heated by means of heat transfer oil. The temperature in the reactor was 72-76° C. In addition, air was passed into the natural circulation evaporator, the amount of air being 2 l/h.

The distillation column had a diameter of 50 mm and a length of 700 mm and was filled with 8 mm glass rings.

After the initially taken mixture circulated in the evaporator, 2 700 g of acrylic acid were metered in over 2 hours. The azeotropic mixture separated off via the column was condensed and the aqueous phase was discharged. The cyclohexane phase was partly (800 g/h) metered into the natural circulation evaporator from below and partly fed as reflux to the distillation column and the internal temperature of the reactor was thus controlled. The minimum reflux of the distillation column was 1 600 g/h. After the addition of acrylic acid, the reaction temperature was increased to 96-99° C. in the course of 2 hours. After an esterification time of 10 hours, the experiment was terminated and the reaction mixture was rapidly cooled to 10° C. The aqueous phase separated off (98% of theory) contained 6.4% of acrylic acid. The reaction mixture (7 320 g) contained 3.5% of acrylic acid.

By adding cyclohexane to the reaction mixture, a crude ester concentration of about 65% was established, and the mixture was cooled to 10° C. and washed with an 8% strength sodium chloride solution (1 500 g) in a stirred container. After the aqueous phase had been separated off, the organic phase was mixed with a 12% strength sodium hydroxide solution until a pH of 13 was obtained, the temperature being kept at 20-30° C.

The neutralized organic phase was mixed with 1 g of hydroquinone monomethyl ether and heated to 60° C. in a stirred container, and the cyclohexane was separated off by distillation (final reduced pressure 100 mbar). The residual cyclohexane content was about 1.5%.

In a packed column (5 mm Raschig rings), the residual cyclohexane was reduced to a content of <0.1% at 70° C. by means of preheated air (60° C., 30 l/h).

The end product (5 010 g) was clear and substantially colorless (Hazen color number 20). The degree of esterification determined by means of the ester number was 99% of theory, the yield was 98% of theory, the acrylic acid content was 0.04%, the cyclohexane 5 content was 0.03% and the viscosity (according to DIN 51562 at 25° C.) was 10 mPa.s.

### EXAMPLE 7

### COMPARATIVE EXAMPLE

The procedure was as in example 6, except that no preliminary washing with the sodium chloride solution was carried out. The copper content in the end product was 15 ppm.

### EXAMPLE 8

2 814 g of dipropylene glycol, 3 g of methoxyphenol in the form of a 2% strength aqueous solution, 6 g of 50% strength hypophosphorous acid, 1 g of copper(II) chloride in the form of a 40% strength aqueous solution, 300 g of 65% strength p-toluenesulfonic acid and 1 700 g of cyclohexane were initially taken in a 10 l reactor having an external natural circulation evaporator, distillation column, condenser and phase separation vessel.

The natural circulation evaporator was a tube-bundle heat exchanger heated by means of heat transfer oil. The temperature in the reactor was 73-76° C. In addition, air was passed into the natural circulation evaporator, the amount of air being 2 l/h.

The distillation column had a diameter of 50 mm and a length of 700 mm and was filled with 8 mm glass rings.

After the initially taken mixture circulated in the evaporator, 3 200 g of acrylic acid were metered in over 2 hours. The azeotropic mixture separated off via the column was condensed and the aqueous phase was discharged. The cyclohexane phase was partly (800 g/h) metered into the natural circulation evaporator from below and partly fed as reflux to the distillation column, and the internal temperature of the reactor was thus controlled. The minimum reflux of the column was 1 600 g/h. The reaction temperature was increased to 95-98° C. in the course of 2 hours. After an esterification time of 8 hours, the experiment was terminated and the reaction mixture was rapidly cooled to 20° C. The aqueous phase separated off (94% of theory) contained 8.9% of acrylic acid. The reaction mixture (9 000 g) contained 6.2% of acrylic acid.

By adding cyclohexane to the reaction mixture, a crude ester concentration of about 70% was established, and the mixture was cooled to 10° C. and washed with an 8% strength sodium chloride solution (1 500 g) in a stirred container. After the aqueous phase had been separated off, the organic phase was mixed with a 12% strength sodium hydroxide solution until a pH of 13 was obtained, the temperature being maintained at 20-30° C.

The neutralized organic phase was mixed with 1 g of hydroquinone monomethyl ether and heated to 60° C. in a stirred container, and the cyclohexane was separated off by distillation (final reduced pressure 100 mbar). The residual cyclohexane content was about 1.5%.

In a packed column (5 mm Raschig rings), the residual cyclohexane was reduced to a content of <0.1% at 70° C. by means of preheated air (60° C., 30 l/h).

The end product (4 970 g) was clear and substantially colorless (Hazen color number <50). The degree of esterification determined by means of the ester number was 99% of theory, the yield was 98% of theory, the acrylic acid content was 0.03%, the cyclohexane content was 0.06% and the viscosity (according to DIN 51562 at 20° C.) was 9 mPa.s.

### EXAMPLE 9

2 000 g of cyclohexane, 3 g of methoxyphenol in the form of a 2% strength aqueous solution, 6 g of 50% strength hypophosphorous acid, 1 g of copper(II) chloride in the form of a 40% strength aqueous solution and 300 g of 65% strength p-toluenesulfonic acid were initially taken in a 10 l reactor having an external natural circulation evaporator, distillation column, condenser and phase separation vessel.

The natural circulation evaporator was a tube-bundle heat exchanger heated by means of heat transfer oil. The temperature in the reactor was 72-76° C. The distillation column had a diameter of 50 mm and a length of 700 mm and was filled with 8 mm glass rings.

After the initially taken mixture circulated in the evaporator, 3 520 g of a trimethylolpropane oxylated with 1 mol/mol of ethylene oxide and 3 mol/mol of propylene oxide and 2 800 g of acrylic acid were metered in. The azeotropic mixture separated off via the column was condensed and the aqueous phase was discharged. The cyclohexane phase was partly (800 g/h) metered into the natural circulation evaporator from below and partly fed as reflux to the distillation column, and the internal temperature of the reactor was thus controlled. The minimum reflux of the column was 1 600 g/h. After the end of the addition, the reaction temperature was increased to 95-99° C. in the course of 2 hours. After an esterification time of 8 hours, the experiment was terminated and the reaction mixture was rapidly cooled to 20° C. The aqueous phase separated off (100% of theory) contained 2.6% of acrylic acid. The reaction mixture (6 935 g) contained 3.2% of acrylic acid.

By adding cyclohexane to the reaction mixture, a crude ester concentration of about 60% was established and washing was effected at 20° C. with an 8% strength sodium chloride solution (1 500 g) in a stirred container. After the aqueous phase had been separated off, the organic phase was mixed with a 12% strength sodium hydroxide solution until a pH of 13 was obtained, the temperature being maintained at 20-35° C. The neutralized organic phase was subsequently washed with a 26% strength sodium chloride solution (1 500 g).

The washed organic phase was mixed with 1 g of hydroquinone monomethyl ether and heated to 60° C. in a stirred container, and the cyclohexane was separated off by distillation (final reduced pressure 100 mbar). The residual cyclohexane content was about 1.5%.

In a packed column (5 mm Raschig rings), the residual cyclohexane was reduced to a content of <0.1% at 80° C. by means of preheated air (75° C., 30 l/h).

The end product (4 180 g) was clear and substantially colorless (Hazen color number 30). The degree of esterification determined by means of the ester number was 96% of theory, the yield was 96% of theory, the acrylic acid content was 0.04%, the cyclohexane content was 0.03% and the viscosity (according to DIN 53229 at 23° C.) was 100 mPa.s.

We claim:

1. A process for the preparation of higher (meth)acrylates by reacting (meth)acrylic acid and the alcohol in the presence of at least one acidic catalyst and at least one polymerization inhibitor and in the presence of a solvent which forms an azeotropic mixture with water and distilling off and condensing the azeotropic mixture, the condensate separating into an aqueous phase and an organic phase, wherein
a) the esterification is carried out in a reactor having a circulation evaporator and
b) in the presence of at least 10% by weight, based on the reaction mixture, of solvent, and either
c1) at least a part of the solvent, if required a part of the catalyst (mixture) and, if required, at least a part of the polymerization inhibitor (mixture) are initially taken in the esterification reactor and heated to the boiling point of the component having the lowest boiling point in the system and, as soon as the circulation is in operation, (meth)acrylic acid and alcohol and, if required, residual catalyst, polymerization inhibitor and solvent are metered in together or separately, or
c2) at least a part of the solvent, if required a part of the catalyst (mixture), if required at least a part of the polymerization inhibitor (mixture) and at least a part of the alcohol are initially taken in the esterification reactor and heated to the boiling point of the component having the lowest boiling point in the system and, as soon as the circulation is in operation, (meth)acrylic acid and, if required, the remaining alcohol and, if required, residual catalyst, polymerization inhibitor and solvent are metered in together or separately, or
c3) at least a part of the solvent, at least a part of the catalyst (mixture), at least a part of the polymerization inhibitor (mixture) and at least a part of the (meth)acrylic acid are initially taken in the esterification reactor and heated to the boiling point of the component having the lowest boiling point in the system and, as soon as the circulation is in operation, the alcohol and, if required, the remaining (meth)acrylic acid and, if required, residual catalyst, polymerization inhibitor and solvent are metered in together or separately, or
c4) at least a part of the solvent, at least a part of the catalyst (mixture), at least a part of the polymerization inhibitor (mixture), at least a part of the alcohol and a part of the (meth)acrylic acid are initially taken in the esterification reactor and heated to the boiling point of the component having the lowest boiling point in the system and, as soon as the circulation is in operation, if required the remaining alcohol and the remaining (meth)acrylic acid and, if required, residual catalyst, polymerization inhibitor and solvent are metered in together or separately.

2. A process as claimed in claim 1, wherein, in step c1), c2) or c3), the catalyst/the catalyst mixture is added before the addition of further substances, after the heating is substantially complete.

3. A process as claimed in claim 1 or 2, wherein the reactor is provided with a natural circulation evaporator.

4. A process as claimed in claim 3, wherein a part of the organic phase of the condensate is fed into the natural circulation evaporator.

5. A process as claimed in any of claims 1 to 4, wherein an inert gas is fed into the natural circulation evaporator.

6. A process as claimed in any of claims 1 to 5, wherein the reaction mixture is prewashed.

7. A process as claimed in any of claims 1 to 6, wherein the reaction mixture is neutralized with aqueous base.

8. A process as claimed in claim 7, wherein the temperature in the neutralization does not exceed 35° C.

9. A process as claimed in any of claims 1 to 8, wherein solvent is stripped from the reaction mixture with an inert gas.

10. A process as claimed in any of claims 1 to 9, wherein at least one compound from the group consisting of hydroquinone, hydroquinone monomethyl ether, 2-tert-butyl-4-methylphenol, 6-tert-butyl-2,4-dimethylphenol, 2,6-di-tert-butyl-4-methylphenol, 2-tert-butylphenol, 4-tert-butylphenol, 2,4-di-tert-butylphenol, 2-methyl-4-tert-butylphenol, 4-tert-butyl-2,6-dimethylphenol, hypophosphorous acid, copper acetate, copper chloride and copper salicylate is used as the polymerization inhibitor (mixture).

11. A reaction mixture which contains substantially trimethylolpropane triacrylate and is obtainable by initially taking at least 10% by weight, based on the sum of trimethylolpropane and acrylic acid, of solvent and at least a part of the polymerization inhibitor (mixture) in a reactor having a circulation, and heating, and metering in trimethylolpropane and acrylic acid in a ratio of 1:3.3-4.5, the acidic catalyst required for the reaction being added before trimethylolpropane and acrylic acid are metered in, the reaction temperature being brought to 70-110° C. and the duration of the reaction being less than 20 hours, then, if required, prewashing, neutralizing and, if required, subsequently washing the reaction mixture and then removing the solvent by distillation and stripping to a content of less than 0.5% by weight, based on the reaction mixture.

12. A process as claimed in claim 1, wherein a reaction mixture containing substantially trimethylolpropane triacrylate is prepared.

13. A reaction mixture which contains substantially trimethylolpropane triacrylate and is obtainable by a process as claimed in claim 12.

14. A reaction mixture which contains substantially trimethylolpropane triacrylate, is obtainable by a process as claimed in claim 12 and has a color number of less than 100 APHA and a viscosity of less than 160 mpa.s and/or an ester number of from 480 to 570.

15. A reaction mixture which contains substantially acrylates of alkoxylated trimethylolpropane and is obtainable by initially taking at least 10% by weight, based on the sum of alkoxylated trimethylolpropane and acrylic acid, of solvent and at least a part of the polymerization inhibitor (mixture) in a reactor having a circulation, and heating, and metering in alkoxylated trimethylolpropane and acrylic acid in a ratio of 1:3.3-4.5, the acidic catalyst required for the reaction being added before alkoxylated trimethylolpropane and acrylic acid are metered in, the reaction temperature being brought to 70-110° C. and the duration of the reaction being less than 20 hours, then, if required, prewashing, neutralizing and, if required, subsequently washing the reaction mixture and then removing the solvent by distillation and stripping to a content of less than 0.5% by weight, based on the reaction mixture.
